(19) United States
(12) Patent Application Publication
(10) Pub. No.: US 2026/0021137 A1
(43) Pub. Date: Jan. 22, 2026

(54) COMPOSITIONS FOR TREATING COLITIS AND/OR PREVENTING COLON CANCER

(71) Applicant: Purdue Research Foundation, West Lafayette, IN (US)

(72) Inventors: Qing Jiang, West Lafayette, IN (US); Yiying Zhao, West Lafayette, IN (US); Cindy H. Nakatsu, West Lafayette, IN (US)

(21) Appl. No.: 19/338,792

(22) Filed: Sep. 24, 2025

Related U.S. Application Data
(62) Division of application No. 17/533,536, filed on Nov. 23, 2021.
(60) Provisional application No. 63/118,109, filed on Nov. 25, 2020.
(60) Provisional application No. 63/134,266, filed on Jan. 6, 2021.

(51) Int. Cl. A61K 35/00; A61K 31/352; A61K 35/744; A61P 1/00; A61P 35/00; C12R 1/225

(52) U.S. Cl. CPC A61K 35/00; A61K 31/352; A61K 35/744; A61P 1/00; A61P 35/00; A61K 2035/115; C12R 2001/225

(57) ABSTRACT

Compositions comprising a plurality of live Lactococcus lacticlactis bacteria and one or more compounds selected from δ-tocotrienol (δTE), γ-tocotrienol (γTE), and/or δTE-13′-carboxychromanol (δTE-13′-COOH, abbr. δTE-13′), and to methods for treating colitis and/or preventing colon cancer live with the novel compositions are disclosed.

### CROSS-REFERENCE TO RELATED APPLICATION

This application is a divisional application of U.S. Patent Application Publication No. 20220160759 (U.S. application Ser. No. 17/533,536), filed on Nov. 23, 2021, which claims the benefit of U.S. Provisional Application No. 63/134,266, filed Jan. 6, 2021 and the U.S. Provisional Application No. 63/118,109, filed Nov. 25, 2020, the contents each or which are incorporated herein entirely.

### TECHNICAL FIELD

The present disclosure relates to novel compositions comprising a plurality of live Lactococcus lactis bacteria and one or more compounds selected from δ-tocotrienol (δTE), γ-tocotrienol (γTE). and/or δTE-13′-carboxychromanol (OTE-13′-COOH. abbr. δTE-13′), and to the method for treating colitis and/or preventing colon cancer live with the novel compositions.

### BACKGROUND

This section introduces aspects that may help facilitate a better understanding of the disclosure. Accordingly, these statements are to be read in this light and are not to be understood as admissions about what is or is not prior art.

Colorectal cancer (CRC) is the third most common cancer worldwide. It is estimated that there are more than 1.2 million new cases and 600,000 deaths from this cancer each year. Because there is no effective treatment for the late-stage disease, chemoprevention with effective and relatively safe compounds to inhibit or delay cancer progression in high-risk people is important for decreasing CRC-associated mortality. Chronic inflammation contributes to CRC and inflammatory bowel disease (IBD) is associated with increased risk of CRC. In particular, up to 20% of IBD patients develop colitis-associated colon cancer (CAC) within 30 years of disease onset, and more than 50% of them will die from the cancer. Therefore, it is important to develop preventive agents with anti-inflammatory and anticancer activities against the deadly CAC.

Natural forms of vitamin E consist of eight lipophilic antioxidants including α-, β-, γ-, δ-tocopherol (αT, βT, γT, δT) and α-, β-, γ-, δ-tocotrienol (αTE, βTE, γTE, δTE). Studies have demonstrated that specific vitamin E forms and metabolites have anti-inflammatory and anticancer effects and exhibit cancer-prevention activities in animal models. In particular, δ- and γ-tocotrienol (δTE, γTE) (FIG. 1A) inhibit endotoxin-stimulated NF-κB and cytokines in macrophages, exhibit anticancer effects against CAC induced by azoxymethane (AOM) and sodium dextran sulfate (DSS) in mice, and appear to be stronger than tocopherols for these effects. Despite these promising results, there are significant knowledge gaps hindering further translation of these basic discoveries to the clinic. In particular, δTE is known to be metabolized to various carboxychromanols including δTE-13′-carboxychromanol (δTE-13′-COOH, abbr. δTE-13′, FIG. 1B). δTE-13′ appears to be the predominant metabolite detected in feces of rodents fed δTE. Importantly, it has been shown that δTE-13′, which is also found in Garcinia kola, is a dual inhibitor of cyclooxygenases and 5-lipoxygenase and inhibits AOM/DSS-induced tumorigenesis in mice. Because δTE-13′ is generated via δTE metabolism in vivo, it is valuable to compare anticancer efficacy between the two for potentially delineating the role of metabolism in tocotrienols' cancer-preventing effects. In addition, although it is recognized that the gut microbiota play significant roles in CRC development, whether δTE or δTE-13′ has any impact on gut microbes during tumorigenesis is not known.

To address these questions, the present disclosure has provided study to evaluate the effect of δTE-rich tocotrienols containing δTE/γTE (8/1, abbreviated as δTE diet) and δTE-13′ on AOM/DSS-induced tumorigenesis and their impact on inflammation, i.e., pro-inflammatory cytokines in mice (study design shown in FIG. 1C). Further, this study has examined the effect of these compounds on gut microbiota using 16S rRNA gene sequencing.

SUMMARY

The present disclosure relates to novel compositions comprising a plurality of live Lactococcus lactis bacteria and one or more compounds selected from δ-tocotrienol (δTE), γ-tocotrienol (γTE), and/or δTE-13′-carboxychromanol (6TE-13′-COOH, abbr. δTE-13′), and to the method for treating colitis and/or preventing colon cancer with the novel compositions.

In one embodiment, the present disclosure provides a composition comprising:
a plurality of live Lactococcus lactis bacteria; and
one or more compounds selected from:

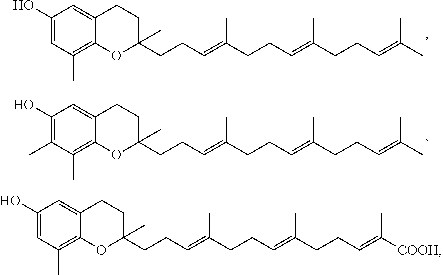

any combination, stereoisomer, tautomer, solvate, and pharmaceutically acceptable salt thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. For brevity/fit in the drawings, AOM/DSS treatment is occasionally abbreviated as AD.

FIG. 1 illustrates (A) The chemical structures of δTE, γTE and δTE-13′; (B) Metabolism of δTE and δTE-13′; and (C) The study design of AOM/DSS model in mice-Six-seven-week old male Balb/c mice were i.p. injected with AOM at 9.5 mg/kg body weight. A week later, AOM-injected mice were randomized into AIN-93G (control), δTE-supplemented and δTE-13′-supplemented groups. Meanwhile, mice were given 1.5% DSS in drinking water for 1 week. The DSS cycle was repeated after a two-week interval.

FIG. 2 illustrates the effect of δTE and δTE-13′ on colon tumorigenesis. (A) Representative photos of AOM/DSS (AD)-induced tumorigenesis in the colon, in comparison with healthy control (non-AD); (B) H&E histology of normal colon tissue and AOM/DSS-induced tumors identified as adenomas in different diet groups; (C) Effects of δTE and δTE-13′ on total tumor multiplicity and large polyps with size >2 mm² (Mean±SEM, n=16) in AD-treated mice; and (D) Effects of δTE and δTE-13′ on log transformed tumor area (Mean±SEM, n=16). Differences among different treatment groups were compared via Kruskal-Wallis followed by Mann-Whitney test. *P<0.05, **P<0.01 indicate difference between control and δTE or δTE-13′-supplement groups in AD-treated animals.

FIG. 3 illustrates the effect of δTE-13′-and δTE on pro-inflammatory cytokines. Immunoplex assay was used to measure cytokines in colon homogenates including granulocyte macrophage colony stimulating factor (GM-CSF) (A), monocyte chemoattractant protein-1 (MCP-1) (B), interleukin-6 (IL-6) (C), interleukin 1β (IL-1β) (D) and tumor necrosis factor α (TNFα) (E). Mann-Whitney test was used to compare AD treatment vs. non-AD or δTE or δTE-13′ (*P<0.05 indicates significant difference). (F)—Significant spearman correlations among multiple factors and cytokines: PAST ver. 3.24 was used for analysis of correlation among cytokines and multiple factors including total tumor #, large-size (>2 mm²) tumor # and ratio of colon length to weight (L/W). The gradient bar represents the correlation coefficient where blue stands for positive correlation and red stands for negative correlation.

FIG. 4 illustrates the effect on gut microbiota composition. (A) Principle coordinates analysis (PCoA) plot of fecal microbiota analyzed with weighted UniFrac matrix. Statistical difference among different groups was analyzed by perMANOVA (p-value is 0.001) and PERMDISP results are insignificant. Blue: healthy control; Red: AD group; Green: AD+δTE; yellow: AD+δTE-13′; and (B) Relative abundances of the predominant phylum Firmicutes and Bacteroidetes in the feces. F/B—the ratio of relative abundance of Firmicutes to Bacteroidetes. Kruskal-Wallis was used for statistical analyses, and values with no common letters differ.

FIG. 5 illustrates differentially abundant taxa identified by ANCOM at the family (A, B), genus (C, D) and species (E, F) level. We performed ANCOM (Analysis of Composition of Microbiomes) to identify differentially abundant taxa (see Materials and Methods, below). Relative abundance (%) of identified taxa was analyzed by Kruskal-Wallis followed by Mann-Whitney tests. Means without a common letter differ (P<0.05).

FIG. 6 illustrates histogram of the LDA scores≥3.5 analyzed by LEfSe. Fecal microbiota data were analyzed by linear discriminant analysis (LDA) effect size (LEfSe), which identified microbes enriched by control diet (red bars), δTE (green bars) or δTE-13′ supplemented group (blue bars) in AOM/DSS treated mice. Based on these results, further statistical analyses were performed to identify difference among groups.

FIG. 7 illustrates the CCA biplot shows the relationships between the gut microbial community and environmental variables. The CCA analysis was performed with relative abundances of fecal microbiome as the species matrix and the environmental variables as the environmental matrix, including AOM/DSS (AD) treatment, δTE or δTE-13′ supplementation, tumor size and colon L/W (length/weight). CCA1 and 2 explained the majority of the total constrained variation of 53% (P=0.002) and 22% (P=0.006), respectively. Variable biplot arrows indicate the direction of environmental gradients. Angles between arrows corresponds to the relationship of the experimental variables to one another based on the relative abundance of fecal microbiome. The relative length of arrows corresponds to the importance of the respective variables in the model. The dots represent the microbial composition of each animal.

FIG. 8 illustrates concentrations of vitamin E forms (A and B) and δTE-13′ and metabolites (C and D) in the plasma and feces and correlation of fecal levels of δTE-13′ with gut microbes (E and F). Vitamin E forms were measured by HPLC, and δTE-13′ and metabolites were quantified by LC-MS/MS, as described under Materials and methods. Student t-test was used for statistical analyses, and *P<0.05, **P<0.01, ***P<0.001 indicate differences between mice fed with control diet and diets supplemented with δTE or δTE-13′. Results are means±SEM (n=5˜6 per group). Panels E and F show Spearman correlations of the relative abundance of fecal Lactococcus or Lachnospiraceae NK4A136 group uncultured bacterium with concentrations of fecal δTE-13′ (combined 3 DB and 2 DB).

FIG. 9 illustrates combining δTE-13′-COOH with live Lactococcus Lactis Subsp. cremoris attenuated colitis-associated damage.

### DETAILED DESCRIPTION

For the purposes of promoting and understanding of the principles of the present disclosure, reference will now be made to embodiments illustrated in drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

### Materials and Methods

### Materials, Reagents and Diets

δTE-13′ (>92%) used in animal studies was isolated from Garcinia kola seeds based on published procedures. See Jang Y, Park N Y, Rostgaard-Hansen A L, Huang J, Jiang Q. Vitamin E metabolite 13′-carboxychromanols inhibit pro-inflammatory enzymes, induce apoptosis and autophagy in human cancer cells by modulating sphingolipids and suppress colon tumor development in mice. Free Radical Biology & Medicine. 2016;95:190-9. Azoxymethane (AOM) was purchased from Sigma and dextran sodium sulfate (DSS, Mw36,000-50,000 Da) was from MP Biochemicals (Solon, OH). δTE/γTE (8/1) was a gift from American River Nutrition (Hadley, MA). AIN-93G diet was used as the control diet. The treatment diets containing δTE/γTE (8/1) and δTE-13′-COOH at 0.035% (˜2.2 μmoles daily) and 0.04% diet (˜2.3 μmoles daily), respectively, were custom-made based on the AIN93G by Dyets Inc (Bethlehem, PA). These doses are equivalent to daily intake of 200 or 230 mg for a 60 Kg adult. All diets were stored at 4° C. in the dark and food given to mice was changed once a week.

### Animal Study and Evaluation of Acute Colitis During DSS Treatment

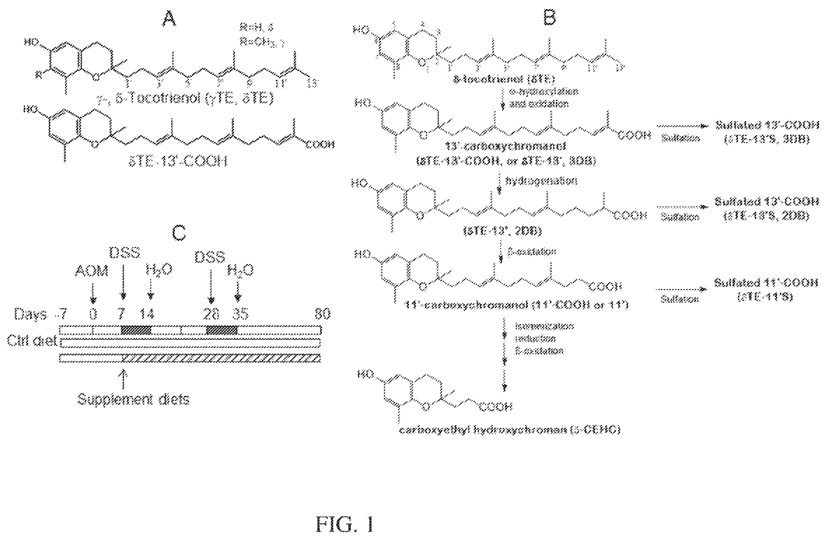

The animal use protocol was approved by the Animal Care and Use Committee at Purdue University. After one-weck adaptation, 6-7 weck old male Balb/c mice from Harlan, (Indianapolis, IN) were i.p. injected with AOM at 9.5 mg/kg body weight. A week later, AOM-injected mice were randomized into AIN-93G (control), δTE-supplemented and δTE-13′-supplemented groups. Meanwhile, mice were given 1.5% DSS in drinking water for 1 week. The DSS cycle was repeated after a two-week interval (design outlined in FIG. 1C). During DSS treatment, we evaluated colitis severity by monitoring body weight, food intake, rectal bleeding and stool consistency. Clinical colitis scores were quantified as follows: bleeding, 0=no blood, 1=feces with blood less than 50%, 2=feces with blood more than 50% but less than 100%, and 3=feces with full blood; stool softness, 0=hard, 1=a bit soft, 2=soft, and 3=very soft.

### Tissue Harvest and Tumor Analysis

During tissue harvest, colons were removed, rinsed with cold PBS, cut open longitudinally, and macroscopically examined. The size and number of macroscopic tumors were measured and recorded. Colons were cut longitudinally in half. Half of the colon was frozen immediately and stored in −80° C. until use and the other half was fixed flat in 4% formaldehyde at 4° C. overnight. Fixed colons were embedded in paraffin, sectioned at 5 μm and stained with Hacmotoxylin and Eosin (H&E).

Extraction of vitamin E forms and their metabolites from the plasma and feces

Vitamin E forms were extracted as previously described. Briefly, plasma tocopherols and metabolites were extracted using a mixture of methanol/hexane (1/2,v/v) in the presence of butylated hydroxytoluene (BHT; 0.8 mM) and ascorbate (0.2 mg/ml). See iang Q, Moreland M, Ames B N, Yin X. A combination of aspirin and gamma-tocopherol is superior to that of aspirin and alpha-tocopherol in anti-inflammatory action and attenuation of aspirin-induced adverse effects. The Journal of Nutritional Biochemistry. 2009;20:894-900. For fecal samples, ˜30 mg feces were smashed and homogenized in 2 ml methanol with BHT (0.8 mM) and ascorbate (0.2 mg/ml). After brief centrifugation, a 1.4 ml methanol layer was obtained and added with 200 μl of PBS, and extracted by 5 ml hexane. For plasma and fecal samples, the hexane and methanol layer were used for quantification of vitamin E forms and metabolites, respectively.

### Quantification of Vitamin E Forms

Different vitamin E forms were separated on a 150×4.6 mm, 5 μm SUPELCOSIL™ LC-18-DB column (Supelco, Bellefonte, PA), and eluted with 95/5 (v/v) methanol/0.1M lithium acetate (final 25 mM, pH 4.75) at a flow rate of 1.3 ml/min. Tocopherols and tocotrienols were monitored by coulometric detection (Model Coulochem II, ESA Inc., Chelmsford, MA) at 300 (upstream) and 500 mV (down-stream electrode) using a Model 5011 analytical cell

### Analysis of Vitamin E Metabolites By Liquid Chromatography Tandem Mass Spectrometry (LC-MS/MS)

The detailed LC-MS/MS method for analyzing vitamin E metabolites has been previously reported. See Jiang Q, Xu T, Huang J, Jannasch AS, Cooper B, Yang C. Analysis of vitamin E metabolites including carboxychromanols and sulfated derivatives using LC/MS/MS. J Lipid Res. 2015;56:2217-25. Briefly, vitamin E metabolites were quantified based on external standards with internal standards for correction of injection and extraction. The LC-MS/MS analysis was done with an Agilent1200 liquid chromatography system coupled to an Agilent 6460 QQQ mass spectrometer equipped with a jet stream electrospray ionization (ESI) source (Santa Clara, CA). The chromatography utilized an Atlantis dC18 column (2.1×150 mm, 3 μm) from Water's Corporation (Milford, MA). Buffer A and B consisted of acetonitrile/ethanol/water (165/135/700, v/v/v) and acetonitrile/ethanol/water (539/441/20, v/v/v), respectively. The LC gradient ran at 0.3 ml/min as follows: 0-1 min, 0% B; at 30 min, 99% B; at 35 min, 99% B; at 37 min, 0% B. Negative polarity ESI was used with gas temperature of 325° C., gas flow at 10 L/min, nebulizer pressure at 30 psi, sheath gas temperature of 250° C., sheath gas flow at 7 L/min, capillary voltage at 4000V, nozzle voltage at 1500 V, and an electron multiplier voltage of −300 V. All data were evaluated with Agilent MassHunter Qualitative Analysis software, versionB.01.06.

### Analyses of Cytokines By Multiplex Assays

Colon tissues were homogenized with POLYTRON® homogenizer (Kinematica AG, Switzerland) on ice in the lysis buffer consisting of 150 mM NaCl, 1 mM EDTA (pH 8.0), 20 mM Tris (pH 7.5), 0.5% Tween 20, 10 uM indomethacin (Cayman), and 1× protease inhibitor cocktail (Sigma). Protein concentrations in tissue homogenates were quantified by BCA assay. The concentrations of selected cytokines were analyzed using a Mouse Cytokine Array Proinflammatory Focused 10-Plex Discovery Assay by Eve Technologies (Calgary, AB Canada). The 10-Plex Discovery Assay includes IFNγ, IL-1β, GM-CSF, IL-2, IL-4, IL-6, IL-10, IL-12p70, MCP-1 and TNFα.

### DNA Extraction

DNAs from ^{˜}30-40 mg fecal samples were isolated using FASTDNA™ SPIN kit for isolating DNA from soil (MP Biomedicals, Solon, Ohio). The quality of the DNAs was assessed by NanoDrop One (260/280 OD ratio) (Thermo Fisher Scientific. Weltham, Mass.) and 0.8% agarose gel electrophoresis. The DNAs were stained with Hoechst 33258 dye and quantities were determined using a NanoDrop 3300 fluorospectrometer (Thermo Fisher Scientific. Weltham, Mass.).

16S rRNA Gene PCR Amplification and Amplicon Sequencing

DNA templates (10 ng) were used for two-step polymerase chain reactions (PCRs) using Q5 High Fidelity DNA Polymerase to minimize errors (New England Biolabs, Ipswich, Mass.). The first PCR reaction is to amplify the 16S rRNA V3 and V4 region (^{˜}460 bp), with forward primer (343-357: 5′ TAC CGR AGG CAG CAG 3′), and a reverse primer (804-790: 5′ CTA CCR GGG TAT CTA ATCC 3′). which is based on primer accuracy and coverage of phylogenetic information that has been determined for short sequencing reads. Four degenerate bases were added to the 1st cycle forward primers to maximize detected microbial diversity and improve the base-calling accuracy of sequencing. The second PCR is to tag the amplicons with unique barcodes using 8-bp tagged forward and reverse primers (Illumina) (support.illumina.com). The number of PCR cycles were 15 in the first step and 5 in the second step to minimize formation of PCR artifacts. After each PCR, unincorporated primers and nucleotides were removed from PCR amplicons using Agencourt AMPure XP kit (Beckman Coulter). The PCR products were evaluated by 1.2% agarose gel and quantified using a NanoDrop 3300 fluorospectrometer after staining with Quantifluor dsDNA Assay Kit (Promega, Madison, Wis.). Amplicons from each sample were combined in equimolar quantities for 250 bp pair-end sequencing using a MiSeq system (Illumina, San Diego, Calif.).

Analysis of the Gut Microbiota Based on 16S rRNA Gene Sequencing Data

The raw sequencing data were processed on the QIIME2 platform. The quality of the sequences was checked and bases with low-quality scores (Q less than 30) were trimmed. After denoising, merging and chimeric-checking using DADA2, amplicon sequence variants (ASVs) were aligned with the SILVA 132 marker gene reference database. After rarefaction, microbial alpha diversities were computed using observed OTUs, Faith Phylogenetic Diversity (PD), Piclou evenness and Shannon indices. For beta diversities, principal coordinate analysis (PCoA) was performed using Unweighted UniFrac, Weighted UniFrac, Jaccard and Bray Curtis distance matrices. Permutational (non-parametric) multivariate statistic (perMANOVA) and permutation analysis of multivariate dispersions (PERMDISP) were used to determine beta diversity significance. To identify differential abundant taxa, we performed analysis of composition of microbiomes (ANCOM) followed by Kruskal-Wallis to determine pair-wise significance. ANCOM takes into account the compositional nature of the dataset and compares absolute abundance in the community. Furthermore, we used linear discriminant analysis (LDA) effect size (LEfSe) to identify microbial taxa that are enriched by the dietary treatments. The cutoff for LDA was set at 3.5. In addition, canonical correspondence analysis (CCA) in PAST3 (Paleontological statistics) was used to determine correlation between relative abundances (at the species level) of gut microbial communities and environmental variables including AOM/DSS treatment, δTE, δTE-13′, ratio of colon length/weight (L/W) and large-size tumors. Significance of the model for the correlations was calculated using a Monte Carlo test with 999 permutations.

### Statistical Analysis

Tumor multiplicity and log transformed tumor area were analyzed by Kruskal-Wallis followed by Mann-Whitney test. Vitamin E and metabolites were compared using Student's t test. For cytokine data, Mann-Whitney test was used to compare two groups including AD vs. non-AD, AD vs. δTE, and AD vs. δTE-13′. PAST 3.24 was used for analysis of Spearman correlation among cytokines and multiple factors. For correlation analysis of gut microbiota and δTE-13′, Spearman correlation tests were performed using RStudio 1.1.456 and adjusted with Benjamini-Hochberg correction to control the false discovery rate for multiple testing. Other statistical analyses are described in figure captions.

### RESULTS

### δTE and δTE-13′ Inhibited AOM/DSS-Induced Colon Tumorigenesis

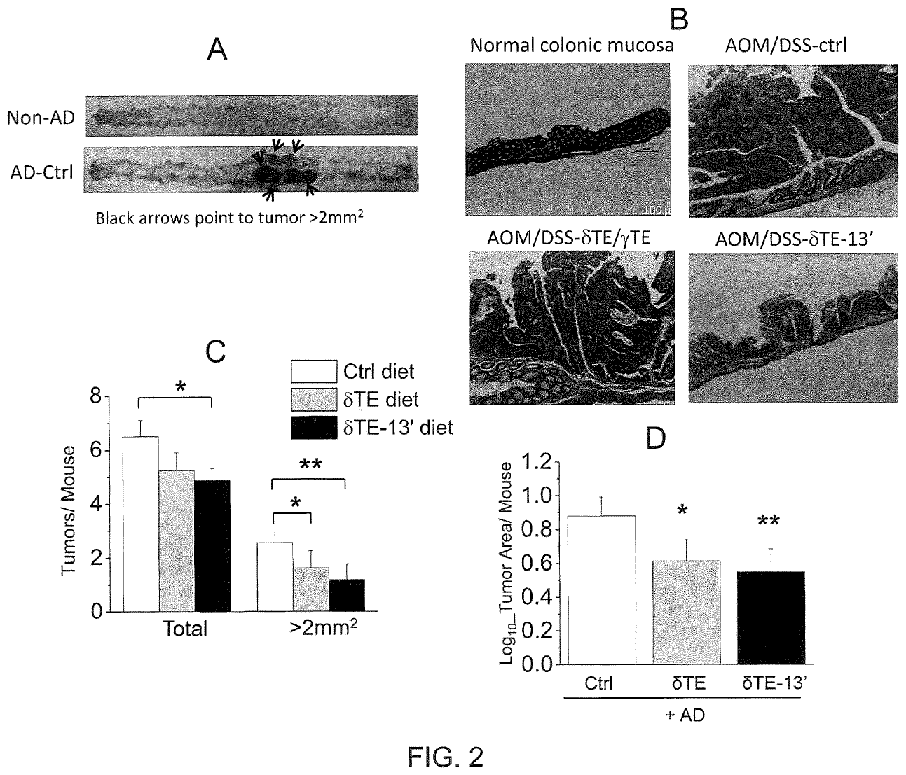

AOM/DSS treatment induced tumorigenesis in the middle to the distal colon (FIG. 2A). Histopathological analyses of tumors from mice fed the control and supplemented diets revealed that all the tumors were adenomas, as indicated in one H&E-stained colon section from each treatment group (FIG. 2B). Compared with the control diet, δTE-13′ supplementation decreased total and large size (>2 mm²) polyps by 25% (p<0.05) and 55% (p<0.01), respectively (FIG. 2C). Supplementation of δTE also inhibited large tumors by 34% (P<0.05), but had no significant impact on total tumor multiplicity compared with controls. In addition, δTE and δTE-13′ supplement decreased tumor surface area by 31% (P<0.05) and 38% (P<0.01), respectively (FIG. 2D). It is noteworthy that supplementation of δTE/γTE or δTE-13′ for more than two months (FIG. 1B) did not affect animals' body weight, food intake or organ weight, indicating lack of obvious adverse effects.

### The Effect on Pro-Inflammatory Cytokines in the Colon

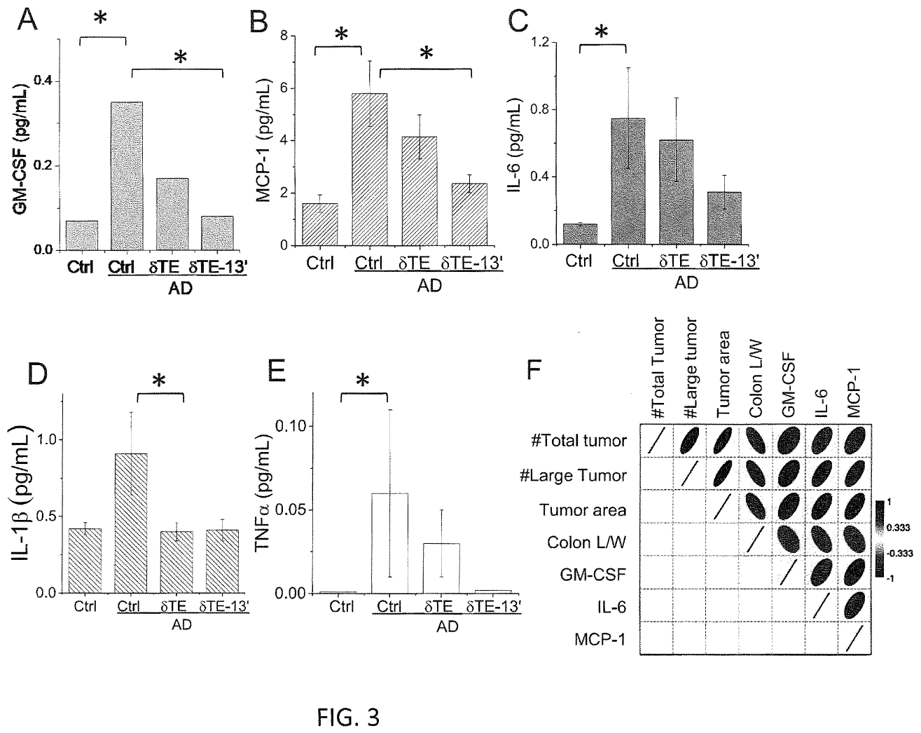

Because inflammation plays a critical role in promotion of colitis-associated colon cancer and both δTE/γTE and δTE-13′ have been shown to have anti-inflammatory properties, we next evaluated potential impact of these compounds on AOM/DSS-induced cytokines in colon homogenates. Compared with healthy controls, AOM/DSS treatment significantly increased GM-CSF, MCP-1, IL-6 and TNFα, and showed tendency in elevation of IL-1β (FIG. 3). Importantly, supplementation of δTE-13′ led to significant reduction of GM-CSF and MCP-1 (FIG. 3A and FIG. 3B), while δTE significantly decreased IL-1β (FIG. 3D). Interestingly, we observed positive association between pro-inflammatory cytokines and the number of total tumors and large-size tumors but inverse association with the ratio of colon length to weight (FIG. 3F), supporting a strong link between inflammation and tumorigenesis in this model.

### δTE/γTE or δTE-13′ Treatment Caused Significant Changes in the Composition (β-Diversity) and Specific Taxa, But Did Not Affect the Richness (α-Diversity) of the Gut Microbiota

The gut microbiota have been recognized as an important regulator of CRC. We next examined the impact of δTE and δTE-13′ supplementation on gut microbes. Using 16S rRNA gene sequencing, we obtained average ˜32,650 pair-end sequences per sample with median amplicon length of 429 base pair after denoising using DADA2. Among analyzed samples, we observed eight major phyla with Firmicutes and Bacteroidetes as the predominant phyla, and identified 173 species.

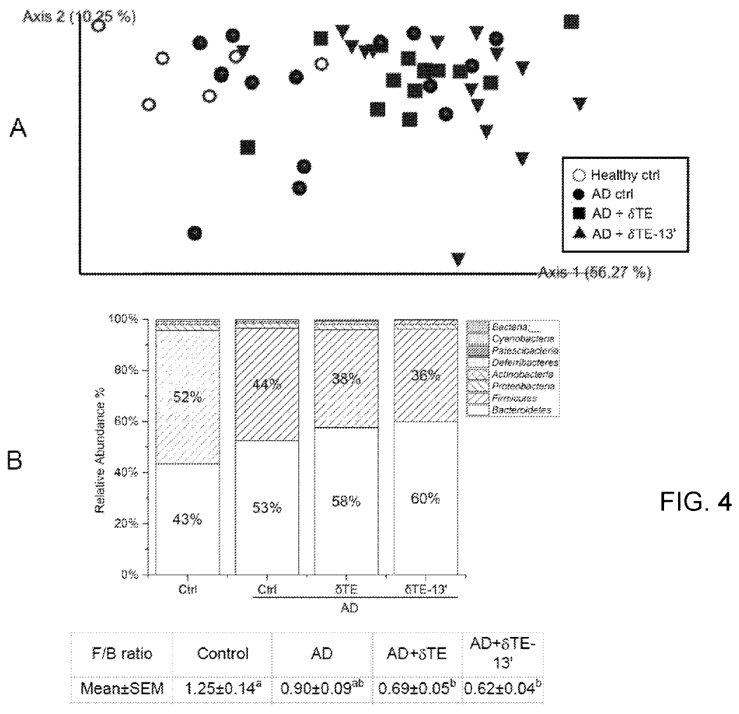

We evaluated the impact of diets and AOM/DSS treatment on α- and β-diversity of the gut microbiota. All the groups had similar number of bacterial species (species richness), although compared with healthy controls, AOM/DSS-treated mice had slightly reduced species evenness, the measure of abundance distribution among species (Table 1). Nevertheless, Shannon index, the overall richness and evenness, was similar among different groups (Table 1). Unlike α-diversity, we observed significant separation of microbial composition among the four treatment groups based on Principle Coordinates Analysis (PCoA) with Jaccard, Bray Curtis, weighted UniFrac or unweighted UniFrac matrices (Table 2) (FIG. 4A). In particular, the gut microbiota in δTE and δTE-13′ groups were closely associated and separated from those from control diet-fed mice. Further, compared with healthy controls, δTE and δTE-13′ supplementation appeared to decrease the ratio of Firmicutes to Bacteroidetes (FIG. 4B). Interestingly, increased ratio of Firmicutes to Bacteroidetes has been associated with obesity.

TABLE 1
The effect on alpha diversity¹ measurements

| Index | Control² | AD³ | AD + δTE⁴ | AD + δTE-13′⁵ |
|---|---|---|---|---|
| Evenness | 0.87 ± 0.01^{a} | 0.83 ± 0.01^{b} | 0.83 ± 0.01^{b} | 0.81 ± 0.01^{b} |
| Observed OTU | 176 ± 18.73^{a} | 200.53 ± 12.26^{a} | 199.81 ± 9.92^{a} | 177.06 ± 8.75^{a} |
| Faith PD | 10.41 ± 0.68^{a} | 10.97 ± 0.31^{a} | 10.95 ± 0.18^{a} | 10.42 ± 0.24^{a} |
| Shannon | 6.48 ± 0.14^{a} | 6.29 ± 0.09^{a} | 6.29 ± 0.06^{a} | 6.05 ± 0.07^{a} |

¹Alpha diversity was caculated upon sequence rarefaction to ensure maximal coverage of taxa.
Evenness index evaluates the relative evenness of the number of microbes detected (1 refers to maximal evenness and 0 refers to maximal unevenness).
Both observed OTU and Faith PD indexes evaluate the absolute microbial richness, while the latter takes phylogenetic diversity into measurement (the higher the richer).
Shannon index is the combination of evenness and richness where community of higher value is considered more diverse.
Different letters indicate significance (p < 0.05) after Kruskal-Wallis post-hoc, and data are presented as mean ± SEM.
²Control: no-cancer healthy control mice fed AIN93G diet, n = 6
³AD: mice treated with azoxymethane (AOM)/dextran sulfate sodium (DSS) and fed AIN93G diet, n = 15
⁴AD + δTE: mice treated with AOM/DSS and fed δTE/γTE-supplemented diet, n = 14
⁵AD + δTE-13′: mice treated with AOM/DSS and fed δTE-13′-supplemented diet, n = 16

TABLE 2
The effect on beta diversity with pairwise statistical comparisons
using different distance matrices

| Distance matrices | Control | AD¹ | AD + δTE² | AD + δTE-13′³ |
|---|---|---|---|---|
| Jaccard | a | b | c | d |
| Bray Curtis | a | b | c | d |
| Unweighted UniFrac | a | b | c | d |
| Weighted UniFrac | a | b | c | c |

Principle Coordinates Analysis (PCoA) was performed.
We used various distance matrices to compare microbial composition (Beta diversity) among different groups including phylogenetic distance matrices unweighted and weighted UniFrac and non-phylogenetic distance matrices including Bray Curtis distance and Jaccard.
Different letters indicate significant difference (p < 0.05) after perMANOVA post-hoc for each matrix.
PERMDISP results for all indices are insignificant.
¹Control: no-cancer healthy control mice fed AIN93G diet, n = 6
²AD: mice treated with azoxymethane (AOM)/dextran sulfate sodium (DSS) and fed AIN93G diet, n = 15
³AD + δTE: mice treated with AOM/DSS and fed δTE/γTE-supplemented diet, n = 14
⁴AD + δTE-13′: mice treated with AOM/DSS and fed δTE-13′-supplemented diet, n = 16

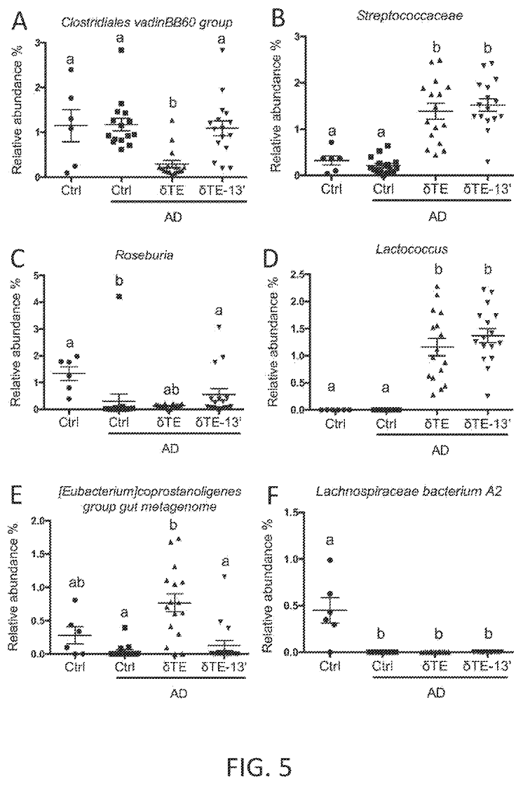

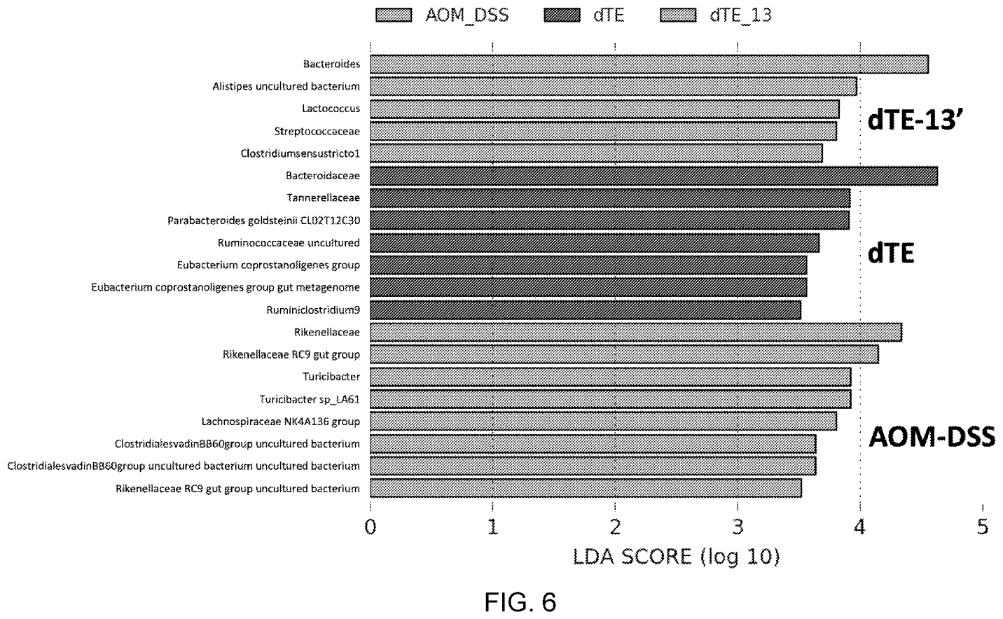

To evaluate the impact of treatments on specific microbes, we used ANCOM and LEfSe analyses. Using ANCOM followed by Kruskal-Wallis, we identified differentially abundant taxa among treatment groups at the family (FIG. 5A, 5B), genus (FIG. 5C, 5D) and species level (FIG. 5E, 5F). In particular, δTE-13′ partially reversed AOM/DSS-associated decrease of genus Roseburia (FIG. 5C). Both δTE-and δTE-13′ supplement significantly elevated the Streptococccaceae family (FIG. 5B) and genus Lactococcus (FIG. 5D) compared with the control diet in either AD-treated or healthy mice. Further, δTE diet uniquely increased [Eubacterium] coprostanoligenes, and decreased Clostridiales vadinBB60 group family compared with other groups. Additionally, the results from LEfSe analysis showed that δTE and δTE-13′ diet enriched Parabacteroides goldsteinii CL02T12C30 and Bacteroides, while other changes caused by these compounds are largely consistent with those identified by ANCOM (FIG. 5 and FIG. 6).

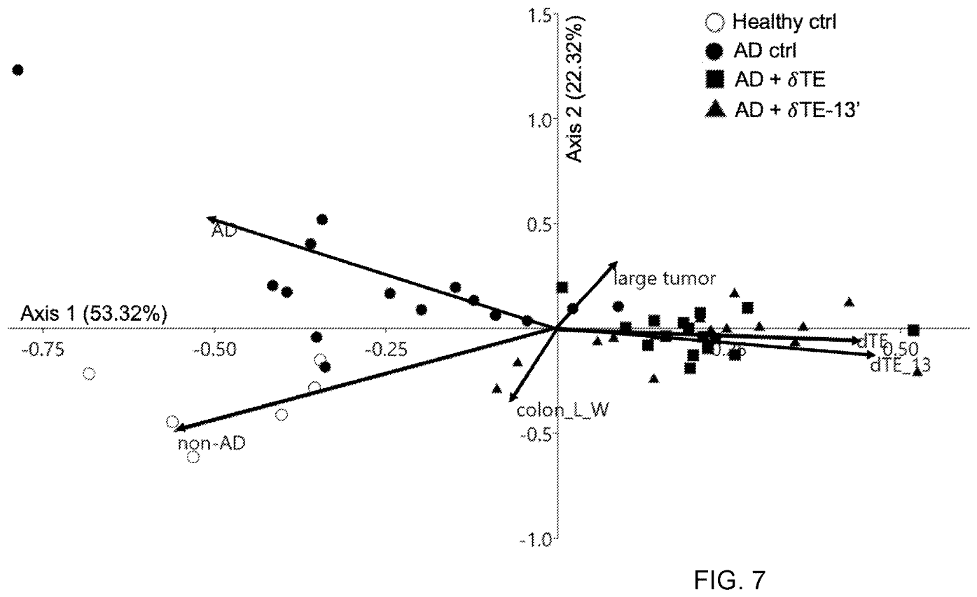

To identify potential factors that influence the gut microbiota, we used canonical correspondence analysis (CCA), a constrained ordination method, to investigate the relationship between relative abundances of the gut microbiota and environmental variables including AOM/DSS treatment, δTE or δTE-13′ supplementation, large-size tumors and colon length/weight (L/W) ratio. As shown in the CCA biplot in FIG. 7, δTE/γTE or δTE-13′ supplementation was the major separating factor on axis 1, which explains 53% of the variations identified in the gut microbiota (P=0.002). These data are consistent with the significant separation of gut microbial composition between mice supplemented with δTE or δTE-13′ and those fed control diet on the PCoA plot (FIG. 4A). On CCA axis 2 that explains 22.3% of the variations, the gut microbiota in healthy controls negatively correlated with those from AD-treated, control diet-fed mice and those with large tumors, indicating that tumorigenesis is the key separation factor. Interestingly, on axis 2, gut microbes in δTE or δTE-13′-supplemented mice were positively associated with those in non-AOM/DSS healthy animals, while negatively correlated with those in control diet and AOM/DSS treated mice or those with large-size tumors (FIG. 7, P=0.006). Overall, these data suggest that δTE and δTE-13′ supplements caused favorable changes of the gut microbiota.

Concentrations of δTE-, δTE-13′ and Metabolites in the Plasma and Feces and Correlation with Gut Microbiota

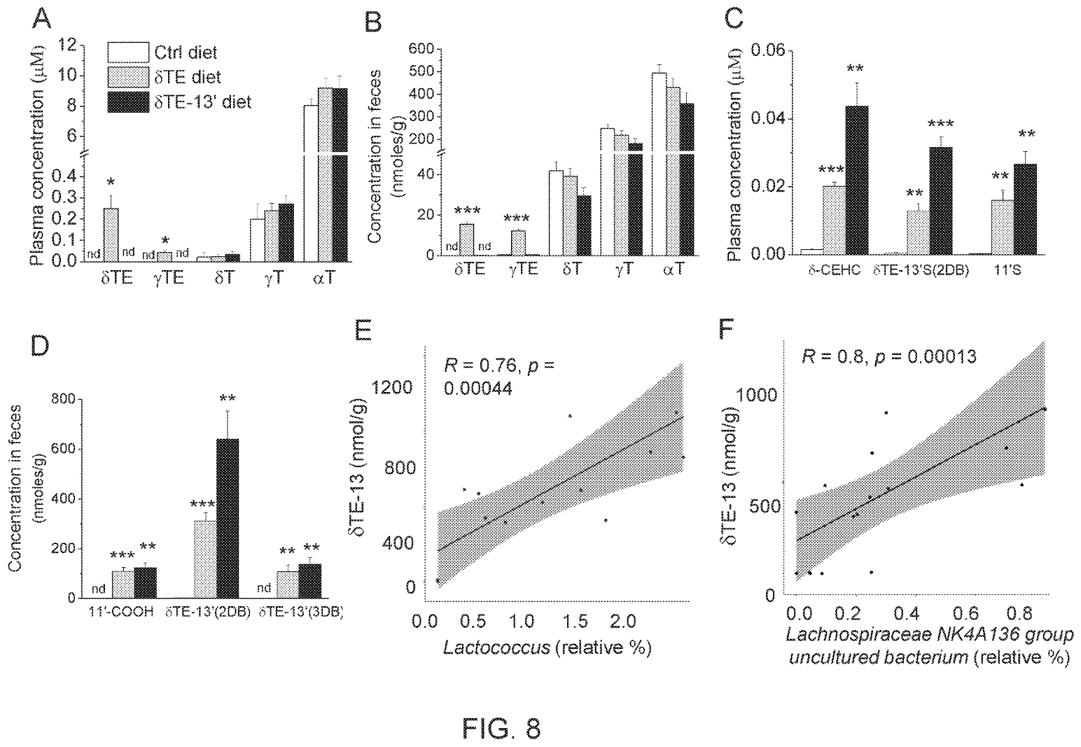

Compared with control diet, supplementation of δTE/γTE significantly increased these tocotrienols in the plasma and feces without affecting tocopherols (FIG. 8A, 8B). Supplementation of δTE/γTE led to elevation of metabolites including δ-CEHC (3,4-dihydro-6-hydroxy-2,7,8-trimethyl-2H-1-benzopyran-2-propanoic acid), sulfated δTE-13′ with 2 double bonds (2 DB) and sulfated δTE-11′ in the plasma (FIG. 8C). In feces, the major metabolites as a result of δTE supplementation included unconjugated δTE-13′ and its 2 DB analog as well as 11′-COOH (FIG. 8D). Interestingly, these metabolites in feces were much higher than un-metabolized δTE (comp FIG. 7B vs. FIG. 7D), indicating extensive metabolism of this compound.

Supplementation of δTE-13′ did not result in detectable amount of the parental compound in the plasma, but led to enhancement of similar metabolites to those from δTE (FIG. 8C). Plasma concentrations of δTE-13′ metabolites were about twice as much as those from δTE. In feces, δTE-13′ supplemented animals had increased δTE-13′ with 3 DB (the parental compound) and metabolites including δTE-13′ with 2 DB (hydrogenated metabolite) and 11′-COOH (a metabolite of β-oxidation) (FIG. 8D). These metabolite profiles indicate that metabolism of δTE-13′ includes side-chain hydrogenation and β-oxidation (FIG. 1B). Interestingly, δTE-13′ with 2 DB as a result of δTE-13′ supplement is significantly higher than that from δTE (FIG. 7D).

Since both δTE and δTE-13′ supplement led to increase of δTE-13′ (3 DB and 2 DB), we performed Spearman correlation analyses between fecal concentrations of δTE-13′ (combining 3 DB and 2 DB) and relative abundance of fecal microbial species. Among 145 taxa with non-zero abundance across the samples, we identified two species showing significant p-value (<0.05) after Benjamini Hochberg correction. As shown in FIGS. 8E and F, genus Lactococcus and Lachnospiraceae NK4A136 group uncultured bacterium were positively associated with fecal δTE-13′ concentration with R value of 0.76 (p<0.001) and 0.8 (p<0.001), respectively.

Combining δTE-13′-COOH with L Lactis Subsp. cremoris Attenuated Colitis-Associated Damage

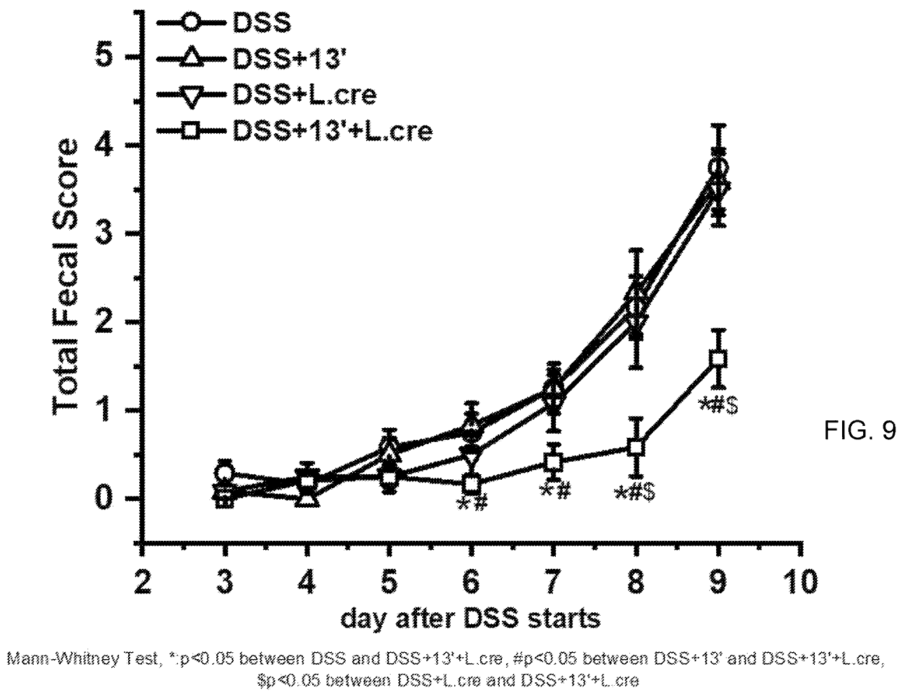

In this disclosure, the effect of combining δTE-13′-COOH with L Lactis Subsp. cremoris on DSS-induced colitis in mice has also been investigated. Male Balb/c mice were divided by four groups: mice fed control diet and gavaged with PBS (DSS), or control diet plus gavage with L Lactis Subsp. cremoris by gavage (DSS+L.cre), or δTE-13′-COOH supplement diet with gavage of PBS (DSS+13′) or δTE-13′-COOH diet plus L Lactis Subsp. cremoris by gavage (13′+L.cre) for 7 days. These mice were then given 2% DSS in drinking water for 9 days to induce experimental colitis. Colitis symptoms (indicated as fecal score in FIG. 9) were evaluated by fecal bleeding and stool consistency. The results indicate that combining δTE-13′-COOH and L Lactis Subsp. cremoris was better than either alone in mitigating colitis, as indicated in FIG. 9. The results of histology evaluation consistently showed that the combination, but not single agent alone, alleviated colitis.

### DISCUSSION

A key finding of our study is that δTE and δTE-13′ at 0.035-0.04% diet, which is equivalent to ˜200-230 mg intake for a 60 Kg adult, significantly suppressed colitis-associated development of large-size adenomas polyps in mice. The observed anticancer effectiveness by δTE-13′ is consistent with our previous study showing that this compound at 0.022% diet significantly suppressed total and large-size tumor multiplicity. It was reported that δTE (δTE/γTE at 8/1) at 0.075% diet suppressed AOM/DSS-induced total tumor multiplicity, but the impact on large polyps was not characterized. In the present study, we observed that δTE at 0.035% diet significantly inhibited large-size polys but did not affect total tumor multiplicity. The discrepancy between our study and previous report is likely caused by different doses of δTE used, which should be further validated in studies examining dose-dependent anticancer effects. Since large-size adenomas have markedly increased risk of developing into malignancy and recurrence in humans compared with small polyps, the ability to inhibit large size adenomas indicates clinically relevant cancer-prevention effectiveness by δTE-13′ and δTE.

Consistent with inhibition of tumorigenesis, δTE-13′ strongly inhibited AOM/DSS-induced pro-inflammatory GM-CSF and MCP-1 and was slightly stronger than δTE/γTE in these effects. On the other hand, δTE/γTE significantly decreased IL-1β. These observations are consistent with previously reported anti-inflammatory effects of δTE-13′ and δTE including inhibition of cyclooxygenases, 5-lipoxygenase and NK-κB. It is well established that proinflammatory cytokines play critical roles in the progression of colon cancer. For instance, GM-CSF has been shown to contribute to inflammation-promoted colon cancer via stimulation of epithelial release of VEGF. Importantly, blockade of this cytokine with anti-GM-CSF resulted in significant reduction of tumor burden in mice. Further, MCP-1 was shown to promote tumorigenesis in Apc^{Min/+} mice; Specifically, compared with wild type mice, MCP-1^{-/-} mice have reduced tumor burden of large-size polyps in the intestine. Additionally, it was demonstrated that strong inflammation mediated by IL-1β is essential for tumor development. Given the causative roles of these cytokines in tumor promotion and progression, the anticancer effects of δTE-13′ and δTE are likely in part rooted in their inhibition of these pro-inflammatory mediators.

An exciting and novel observation is that δTE and its metabolite δTE-13′ are capable of modulating the gut microbiota in the AOM/DSS colon cancer model. δTE or δTE-13′ treatment caused significant changes in microbial β-diversity compared to the control diet. These compounds altered gut bacteria at the family, genus and species level including elevation of the relative abundance of genus Lactococcus and Bacteroides. While causing similar changes to many gut microbes, δTE and δTE-13′ showed differential impact on specific bacteria. For instance, δTE, but not δTE-13, significantly increased [Eubacerium] coprostanoligenes. It is worth mentioning that tocopherols including αT or γT do not share the same modulatory effect on gut microbes as δTE and δTE-13′ (unpublished data by Liu and Jiang). These observations indicate that alteration of gut microbes by vitamin E forms is not likely rooted in their antioxidant activity.

Although our present study does not prove a causative relation between modulation of gut microbiota and suppression of tumorigenesis, specific microbes enhanced by δTE-and δTE-13′ including Lactococcus, Bacteroides and Roseburia are potentially beneficial to mitigating colitis and preventing colon cancer. In particular, Lactococcus lactis subsp. cremoris has been shown to exert cytoprotection and anti-inflammatory effects on colitis in mice. A catalase-producing Lactococcus lactis is reported to inhibit chemically-induced colon cancer in rodents. Human symbiont Bacteroides fragilis protects animals from experimental colitis induced by Helicobacter hepaticus. Moreover, δTE-13′ significantly reversed AOM-DSS-caused depletion of Roseburia, which is reported to be decreased in the stool of patients with inflammatory bowel diseases. Decrease of Roseburia has recently been associated with increased risk of inflammatory bowel diseases. In addition, δTE significantly increased [Eubacerium] coprostanoligenes and Parabacteroides goldsteinii CL02T12C30, microbes that are known to be involved in cholesterol metabolism and enhance intestinal integrity, respectively. These observations indicate that δTE and δTE-13′ supplementation caused healthy changes of gut microbiota, which is supported by the CCA results where on axis 2, gut microbes in δTE and δTE-13′-supplemented mice positively correlate with those in non-AD healthy control mice.

Our current findings provide interesting insights into potential role of metabolites in δTE's anticancer effects and modulation of gut microbes. For instance, the concentrations of δTE-13′ and its hydrogenated metabolite are higher in δTE-13′-supplemented mice than those in δTE-fed animals. Since δTE-13′ has anti-inflammatory and anticancer effect and inhibits GM-CSF and MCP-1 more strongly than δTE as indicated in this study, we argue that δTE-13′ (3 DB and 2 DB) likely contributes to δTE's anti-tumorigenesis effect in vivo. In addition, δTE-13′ and its metabolite may partially contribute to δTE-mediated modulation of the gut microbiota. Specifically, since both δTE and δTE-13′ supplements induced elevation of Lactococcus, this effect is not dependent upon δTE as δTE-13′ and metabolites are generated as a result of δTE metabolism in mice. Consistently, we observed strong correlation between Lactococcus and the concentrations of combined δTE-13′ and its DB metabolite. In contrast, δTE showed unique modulation of gut microbes, which is independent of its metabolites, including increase of [Eubacterium] Coprostanoligenes and decrease of Clostridiales.

The present disclosure demonstrates that δTE-13′ and δTE inhibited colitis-associated colon cancer and δTE-13′ appeared to be stronger than its precursor in suppressing tumor-promoting GM-CSF and MCP-1 in mice. We also discovered that δTE-13′ and δTE are capable of modulating gut microbes, and thus revealed activities of these compounds beyond recognized antioxidant and anti-inflammatory effects. While these data have uncovered intriguing new activities of these promising cancer-preventive agents, our research raises new questions that warrants future investigation. In particular, the nature of how δTE-13′ and δTE interact with gut microbes is not clear and needs to be explored including evaluation of whether these compounds can alter gut microbiota under the non-disease condition. Further, it remains to be determined whether modulation of gut microbes plays a causative role in δTE or δTE-13′-mediated anticancer and anti-inflammatory. In addition, since high amounts of fecal metabolites were detected from δTE and δTE-13′ supplemented mice, it is possible that gut microbes play a role in metabolism of these compounds, which is implicated by a recent publication where antibiotic treatment resulted in decrease of detected metabolite in tissues.

Furthermore, the present disclosure provides that combining δTE-13′-COOH with L Lactis Subsp. cremoris attenuated colitis-associated damage.

In one embodiment, the present disclosure provides a composition comprising:
a plurality of live Lactococcus lactis bacteria; and
one or more compounds selected from:

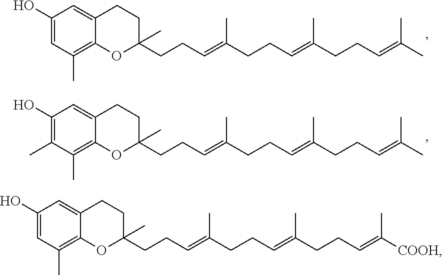

any combination, stereoisomer, tautomer, solvate, and pharmaceutically acceptable salt thereof.

In one embodiment regarding the composition of the present disclosure, the composition comprises:

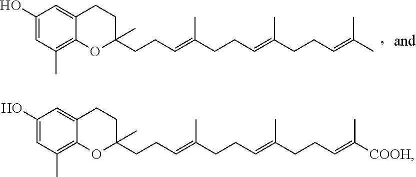 [SMILES: CC(C)=CCC/C(C)=C/CC/C(C)=C/CCC1(C)CCc2cc(O)cc(C)c2O1 ; C/C(=C\CC/C(C)=C/CCC1(C)CCc2cc(O)cc(C)c2O1)CC/C=C(\C)C(=O)O]

or any pharmaceutically acceptable salt thereof.

In one embodiment regarding the composition of the present disclosure, wherein the composition comprises

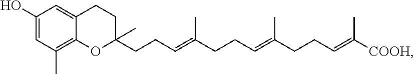 [SMILES: C/C(=C\CC/C(C)=C/CCC1(C)CCc2cc(O)cc(C)c2O1)CC/C=C(\C)C(=O)O]

or any pharmaceutically acceptable salt thereof.

In one embodiment regarding the composition of the present disclosure, wherein said live Lactococcus lactis bacteria has a concentration range from 0.5×10⁸ colony-forming units (CFU) to 5×10¹⁴ CFU.

In one embodiment regarding the composition of the present disclosure, wherein said one or more compounds have a total daily dose amount of 50-500 mg.

In one embodiment regarding the composition of the present disclosure, wherein said live Lactococcus lactis bacteria comprise live Lactococcus lactis Subsp. cremoris.

In one embodiment regarding the composition of the present disclosure, wherein the composition further comprises α-tocopherol, β-tocopherol, γ-tocopherol, δ-tocopherol, any pharmaceutically acceptable salt, or any combination thereof.

In one embodiment regarding the composition of the present disclosure, wherein said α-tocopherol, β-tocopherol, γ-tocopherol, δ-tocopherol, or any combination thereof have a daily dose amount of 50-300 mg.

In one embodiment regarding the composition of the present disclosure, wherein said composition is used for treating colitis and/or preventing colon cancer.

Those skilled in the art will recognize that numerous modifications can be made to the specific implementations described above. The implementations should not be limited to the particular limitations described. Other implementations may be possible.

1. A method for treating colitis and/or colon cancer, said method comprising:
a) diagnosing a patient susceptible to or currently exhibiting colitis and/or colon cancer, and
b) administering a composition comprising:
a plurality of live Lactococcus lactis bacteria at a concentration ranging from 0.5×10⁸ colony-forming units (CFU) to 5×10¹⁴ CFU; and
one or more compounds selected from the group including: 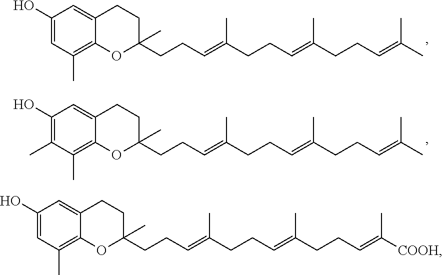
a stereoisomer thereof, a tautomer thereof, a solvate thereof, or a pharmaceutically salt thereof.

2. The method of claim 1, wherein said one or more compounds have a total daily dose amount of 50-500 mg.

3. The method of claim 1, wherein said live Lactococcus lactis bacteria comprise live Lactococcus lactis Subsp. cremoris.

4. The composition of claim 1, wherein the composition further comprises α-tocopherol, β-tocopherol, γ-tocopherol, δ-tocopherol, any pharmaceutically acceptable salt thereof, or any combination thereof.

5. The composition of claim 1 comprising an amount of 50-300 mg of α-tocopherol, β-tocopherol, γ-tocopherol, δ-tocopherol, or any combination thereof.
